(19) United States
(12) Patent Application Publication
(10) Pub. No.: US 2010/0317685 A1
(43) Pub. Date: Dec. 16, 2010

(54) N-PHENYL-IMIDAZO[1,2-a]PYRIDINE-2-CARBOXAMIDE DERIVATIVES, THEIR PREPARATION AND THEIR THERAPEUTIC APPLICATION

(75) Inventor: Jean-Francois PEYRONEL, Palaiseau (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/828,369

(22) Filed: Jul. 1, 2010

Related U.S. Application Data
(63) Continuation of application No. PCT/FR2008/001833, filed on Dec. 31, 2008.

(30) Foreign Application Priority Data
Jan. 2, 2008 (FR) 0800002

(51) Int. Cl. A61K 31/437; C07D 471/04; A61P 25/28; A61P 29/00; A61P 35/00; A61P 25/16; A61P 25/00

(52) U.S. Cl. 514/300; 546/121

(57) ABSTRACT

Compounds of formula (I):

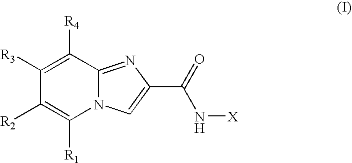

wherein:
X, R₁, R₂, R₃, and R₄ are as defined in the disclosure, and therapeutic uses thereof.

The present invention relates to imidazo[1,2-a]pyridine-2-carboxamide derivatives, to their preparation and to their therapeutic application in the treatment or prevention of diseases involving Nurr-1 nuclear receptors, also known as NR4A2, NOT, TINUR, RNR-1 and HZF3.

A subject-matter of the present invention is the compounds of formula (I):

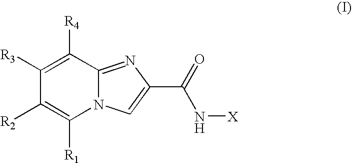

in which:
X represents a phenyl group substituted by a cyano, a (C₁-C₆)alkoxycarbonyl, a (C₁-C₆)alkoxy substituted by one or more halogens or (C₁-C₆)alkyl substituted by one or more halogens, the phenyl group optionally being substituted a second time by a halogen;
R₁ represents a hydrogen atom, a halogen, a (C₁-C₆)alkoxy group, a (C₁-C₆)alkyl group or an NRaRb group, it being possible for the alkyl and alkoxy groups to be optionally substituted by one or more halogen, hydroxyl, amino, or (C₁-C₆)alkoxy group;
R₂ represents one of the following groups:
   a hydrogen atom,
   a (C₁-C₆)alkyl group optionally substituted by one or more groups chosen, independently of one another, from a hydroxyl, a halogen, an amino, an NRaRb group, a (C₁-C₆)alkoxy group or a phenyl group,
   a (C₁-C₆)alkoxy group optionally substituted by one or more groups chosen, independently of one another, from hydroxyl, halogen, amino or NRaRb group,
   a (C₂-C₆)alkenyl group,
   a (C₂-C₆)alkynyl group,
   a —CO—R₅ group,
   a —CO—NR₆R₇ group,
   a —CO—O—R₈ group,
   an —NR₉—CO—R₁₀ group,
   an —N₁₁R₁₂ group,
   an —N═CH—NRaRb group,
   a halogen atom,
   a cyano, nitro, hydroxyiminoalkyl or an alkoxyiminoalkyl group,
   a (C₁-C₆)alkylthio group,
   a (C₁-C₆)alkylsulphinyl group,
   a (C₁-C₆)alkylsulphonyl group,
   a ((C₁-C₆)alkyl)₃silylethynyl group,
   an —SO₂—NR₉R₁₀ group,
   a phenyl group optionally substituted by one or more groups chosen, independently of one another, from the following atoms or groups: halogen, (C₁-C₆)alkoxy, cyano, NRaRb, —CO—R₅, —CO—NR₆R₇, —CO—O—R₈ or (C₁-C₆)alkyl optionally substituted by one or more hydroxyl or NRaRb groups;
R₃ represents a hydrogen atom, a (C₁-C₆)alkyl group, a (C₁-C₆)alkoxy group or a halogen atom;
R₄ represents a hydrogen atom, a (C₁-C₄)alkyl group, a (C₁-C₄)alkoxy group or a fluorine atom;
R₅ represents a hydrogen atom, a phenyl group or a (C₁-C₆)alkyl group;
R₆ and R₇, which are identical or different, represent a hydrogen atom or a (C₁-C₆)alkyl group or form, with the nitrogen atom, a 4- to 7-membered ring optionally including another heteroatom chosen from N, O or S;
R₈ represents a (C₁-C₆)alkyl group;
R₉ and R₁₀, which are identical or different, represent a hydrogen atom or a (C₁-C₆)alkyl group;
R₁₁ and R₁₂, which are identical or different, represent a hydrogen atom or a (C₁-C₆)alkyl group or form, with the nitrogen atom, a 4- to 7-membered ring optionally including another heteroatom chosen from N, O or S;
Ra and Rb represent, independently of one another, a hydrogen atom or a (C₁-C₆)alkyl group or form, with the nitrogen atom, a 4- to 7-membered ring;
with the exception of the compounds where R₁ represents a methyl group or R₂ represents a chlorine atom or R₃ represents a methyl group,
in the form of the base or of an addition salt with an acid,
with the exception of the compounds:
N-(3-chloro-4-cyanophenyl)imidazo[1,2-a]pyridine-2-carboxamide,
methyl 3-({[imidazo[1,2-a]pyridin-2-yl]carbonyl}amino)benzoate,
methyl 2-({[imidazo[1,2-a]pyridin-2-yl]carbonyl}amino)benzoate,
N-[2-(difluoromethoxy)phenyl]imidazo[1,2-a]pyridine-2-carboxamide,
N-[3-(trifluoromethoxy)phenyl]-6-(dimethylamino)imidazo[1,2-a]pyridine-2-carboxamide,
N-[3-(difluoromethoxy)phenyl]-6-(dimethylamino)imidazo[1,2-a]pyridine-2-carboxamide, and
N-[3-(trifluoromethyl)phenyl]-6-(dimethylamino)imidazo[1,2-a]pyridine-2-carboxamide.

WO 2008/003856 discloses compounds having an activity with regard to the abovementioned receptors. The compounds N-[3-(trifluoromethoxy)phenyl]-6-(dimethylamino)imidazo[1,2-a]pyridine-2-carboxamide (Example 76), N-[3-(difluoromethoxy)-phenyl]-6-(dimethylamino)imidazo[1,2-a]pyridine-2-carboxamide (Example 82), and N-[3-(trifluoromethyl)phenyl]-6-(dimethylamino)imidazo[1,2-a]pyridine-2-carboxamide (Example 83) are specifically excluded from the general formula (I) according to the invention.

In addition, the compounds N-(3-chloro-4-cyanophenyl)imidazo[1,2-a]pyridine-2-carboxamide (database accession No. 924038-88-0), methyl 3-({[imidazo[1,2-a]pyridin-2-yl]carbonyl}amino)benzoate (No. 924031-00-5), methyl 2-({[imidazo[1,2-a]pyridin-2-yl]carbonyl}amino)benzoate (No. 924102-15-8) and N-[2-(difluoromethoxy)phenyl]imidazo[1,2-a]pyridine-2-carboxamide (No. 924040-86-8), for which no pharmacological or therapeutic activity has been demonstrated, are also specifically excluded from the said formula (I) according to the invention.

The compounds of formula (I) can comprise one or more asymmetric carbon atoms. They can thus exist in the form of enantiomers or diastereosiomers. These enantiomers or diastereoisomers and their mixtures, including racemic mixtures, come within the invention.

The compounds of formula (I) can exist in the form of bases or of addition salts with acids. Such addition salts come within the invention.

These salts can be prepared with pharmaceutically acceptable acids but the salts of other acids, for example of use in the purification or the isolation of the compounds of formula (I), also come within the invention.

The compounds of formula (I) can also exist in the form of hydrates or solvates, namely in the form of combinations or associations with one or more molecules of water or with a solvent. Such hydrates or solvates also come within the invention.

In the context of the present invention:
a halogen atom is understood to mean a fluorine, a chlorine, a bromine or an iodine;
an alkyl group is understood to mean a saturated, linear, branched or cyclic, aliphatic group which is optionally substituted by a saturated, linear, branched or cyclic, alkyl group. Mention may be made, by way of examples, of the methyl, ethyl, propyl, isopropyl, butyl, isobutyl, tert-butyl, cyclopropyl, cyclobutyl, cyclopentyl, cyclohexyl or methylcyclopropyl groups, and the like;
an alkenyl group is understood to mean a mono- or polyunsaturated, linear or branched, aliphatic group comprising, for example, one or two ethylenic unsaturations;
an alkoxy group is understood to mean an —O-alkyl where the alkyl group is as defined above;
an alkynyl group is understood to mean a mono- or polyunsaturated, linear or branched, aliphatic group comprising, for example, one or two ethylynic unsaturations.

According to another of its aspects, a subject-matter of the present invention is the compounds of formula (I) for which X and R₁ to R₄ are as defined above, it being understood that at least one of R₁, R₂, R₃ and R₄ is other than a hydrogen atom, in the form of the base or of an addition salt with an acid,

with the exception of the compounds where R₁ represents a methyl group or R₂ represents a chlorine atom or R₃ represents a methyl group,
with the exception of the compounds:
N-[3-(trifluoromethoxy)phenyl]-6-(dimethylamino)imidazo[1,2-a]pyridine-2-carboxamide,
N-[3-(difluoromethoxy)phenyl]-6-(dimethylamino)imidazo[1,2-a]pyridine-2-carboxamide, and
N-[3-(trifluoromethyl)phenyl]-6-(dimethylamino)imidazo[1,2-a]pyridine-2-carboxamide.

According to another of its aspects, a subject-matter of the present invention is the compounds of formula (I) in which X and R₁ to R₄ are as defined above, it being understood that at least one of R₁, R₂, R₃ and R₄ is other than a hydrogen atom, in the form of the base or of an addition salt with an acid,

with the exception of the compounds where R₁ represents a methyl group or R₂ represents a chlorine atom or R₃ represents a methyl group,
with the exception of the compounds for which R₂ represents an N-dimethyl group and X represents:
a phenyl group substituted by a methoxy group itself substituted by two or three fluorine atoms, or
a phenyl group substituted by a methyl group itself substituted by three fluorine atoms.

According to another of its aspects, another subject-matter of the present invention is a first group of compounds of formula (I) in which:

X represents a phenyl group substituted by a cyano, a (C₁-C₆)alkoxycarbonyl or a (C₁-C₆)alkoxy substituted by several halogens, the said phenyl group optionally being substituted a second time by a halogen;
R₂ represents an —NR₁₁R₁₂ group or a phenyl group substituted by a (C₁-C₆)alkyl group itself substituted by a hydroxyl group;
R₁, R₃ and R₄ represent a hydrogen atom;
R₁₁ and R₁₂, which are identical or different, represent a (C₁-C₆)alkyl group;
in the form of the base or of an addition salt with an acid,
with the exception of the compounds where R₁ represents a methyl group or R₂ represents a chlorine atom or R₃ represents a methyl group,
with the exception of the compounds:
N-[3-(trifluoromethoxy)phenyl]-6-(dimethylamino)imidazo[1,2-a]pyridine-2-carboxamide and
N-[3-(difluoromethoxy)phenyl]-6-(dimethylamino)imidazo[1,2-a]pyridine-2-carboxamide.

According to yet another of its aspects, the subject-matter of the present invention is a second group of compounds of formula (I) in which:

X represents a phenyl group substituted by a cyano, CO₂Me or OCHF₂ and optionally substituted a second time by a fluorine atom;
R₁, R₃ and R₄ represent a hydrogen atom;
R₂ represents an N-dimethyl group or a phenyl group substituted by a hydroxymethyl group;
in the form of the base or of an addition salt with an acid,
with the exception of N-[3-(difluoromethoxy)phenyl]-6-(dimethylamino)imidazo[1,2-a]pyridine-2-carboxamide.

Mention may in particular be made, among the compounds of formula (I) which are subject-matters of the invention, of the following compounds:
N-(3-cyano-5-fluorophenyl)-6-[3-(hydroxymethyl)phenyl]imidazo[1,2-a]pyridine-2-carboxamide,
N-(3-cyanophenyl)-6-(dimethylamino)imidazo[1,2-a]pyridine-2-carboxamide,
Methyl 3-({[6-(dimethylamino)imidazo[1,2-a]pyridin-2-yl]carbonyl}amino)benzoate,
N-[3-(difluoromethoxy)phenyl]-6-[3-(hydroxymethyl)phenyl]imidazo[1,2-a]pyridine-2-carboxamide,
N-(3-cyanophenyl)-6-[3-(hydroxymethyl)phenyl]imidazo[1,2-a]pyridine-2-carboxamide,
N-(4-cyano-2-fluorophenyl)-6-(dimethylamino)imidazo[1,2-a]pyridine-2-carboxamide,
N-(2-cyano-3-fluorophenyl)-6-(dimethylamino)imidazo[1,2-a]pyridine-2-carboxamide,
N-(2-cyano-3-fluorophenyl)-6-[3-(hydroxymethyl)phenyl]imidazo[1,2-a]pyridine-2-carboxamide,
N-(3-cyano-5-fluorophenyl)-6-(dimethylamino)imidazo[1,2-a]pyridine-2-carboxamide, and their addition salts with an acid.

In accordance with the invention, the compounds of general formula (I) can be prepared according to the process described in Scheme 1.

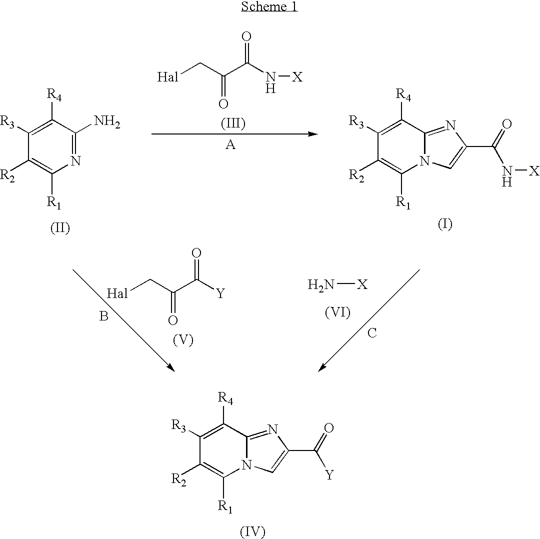

Route A consists in preparing the 2-aminopyridines of formula (II) according to methods known to a person skilled in the art and in forming the imidazo[1,2-a]pyridine ring by condensation with a 2-oxo-N-arylpropionamide derivative (III), in which Hal represents a chlorine, bromine or iodine atom and X is defined as above, by analogy with the methods described by J-J. Bourguignon et al. in Aust. J. Chem., 50, 719 (1997), and by J. G. Lombardino in J. Org. Chem., 30, 2403 (1965), for example. The halogenated derivatives of 2-oxo-N-arylpropionamide (III) can be obtained according to the method described by R. Kluger et al. in J. Am. Chem. Soc., 106, 4017 (1984).

The second synthetic route, B and C, consists in coupling an imidazopyridine-2-carboxylic acid or one of its derivatives of formula (IV), in which Y represents a hydroxyl group, a halogen atom or a (C₁-C₆)alkoxy group, to an arylamine X—NH₂ (VI), in which X is defined as above, according to methods known to a person skilled in the art. Thus, the acid can be converted beforehand to one of its reactive derivatives, such as acid halide, anhydride, mixed anhydride or activated ester, and then reacted with the amine (VI) in the presence of a base, such as diisopropylethylamine, triethylamine or pyridine, in an inert solvent, such as THF, DMF or dichloromethane. The coupling can also be carried out in the presence of a coupling agent, such as CDI, EDCI, HATU or HBTU, under the same conditions without isolation of reactive intermediate. Alternatively, the amine (VI) can be reacted with an ester of the acid of formula (IV) in the presence of a catalyst, such as trimethylaluminium, according to the method of Weinreb, S. et al. (Tet. Lett., 18, 4171 (1977)), or zirconium tert-butoxide. The imidazopyridine-2-carboxylic acids and their derivatives of formula (IV) can be obtained by condensing the appropriate 2-aminopyridines with an ester of the 3-halo-2-oxopropionic acid according to the method described by J. G. Lombardino in J. Org. Chem., 30(7), 2403 (1965), and by then deprotecting the ester to give an acid and, if appropriate, converting the acid to one of its derivatives.

The products of formula (I) and their precursors of formula (II) or (IV) can be subjected, if desired and necessary, in order to obtain products of formula (I) or to be converted to other products of formula (I), to one or more of the following transformation reactions, in any order:

a) a reaction for the esterification or amidation of an acid functional group,
b) a reaction for the amidation of an amine functional group,
c) a reaction for the hydrolysis of an ester functional group to give an acid functional group,
d) a reaction for the transformation of a hydroxyl functional group to an alkoxy functional group,
e) a reaction for oxidation of an alcohol functional group to give an aldehyde or ketone functional group,
f) a reaction for the transformation of aldehyde or ketone functional groups to give an alcohol functional group by reduction or by the action of an organometallic compound, such as an organomagnesium compound,
g) a reaction for the conversion of aldehyde or ketone functional groups to give an oxime derivative,
h) a reaction for the transformation of a nitrile radical to give an aldehyde functional group,
i) a reaction for the transformation of a nitrile radical to give a ketone functional group,
j) a reaction for the oxidation of an alkenyl group to give an aldehyde or ketone functional group,
k) a reaction for the olefination of an aldehyde or ketone functional group to give an alkenyl group,
l) a reaction for the dehydration of a hydroxyalkyl group to give an alkenyl group,
m) a reaction for the total or partial hydrogenation of an alkenyl or alkynyl group to give an alkenyl or alkyl group,
n) a reaction for the catalytic coupling of an organometallic derivative, such as a boron, tin or silicon derivative, with a halogenated derivative in order to introduce an alkyl, alkenyl, alkynyl or aryl substituent,
o) a reaction for the reduction of a nitro group to give a primary amino group,
p) a reaction for the conversion of a primary or secondary amino group to a secondary or tertiary amino group by reductive amination or alkylation,
q) a reaction for the conversion of a primary amino group to an amidine group,
r) a reaction for the oxidation of a thioether functional group to give a sulphoxide or sulphone functional group,
s) a reaction for the protection of the reactive functional groups,
t) a reaction for the removal of the protective groups which the protected reactive functional groups may carry,
u) a reaction for salification by an inorganic or organic acid or by a base in order to obtain the corresponding salt,
v) a reaction for the resolution of the racemic forms to give enantiomers,
the said products of formula (I) thus obtained being, if appropriate, in all the possible isomeric forms, racemic, enantiomeric, diastereoisomeric and tautomeric.

In Scheme 1, the starting compounds and the reactants, when their method of preparation is not described, are commercially available or described in the literature or else can be prepared according to methods which are described therein or which are known to a person skilled in the art.

The following examples describe the preparation of some compounds in accordance with the invention. These examples are not limiting and serve only to illustrate the present invention. The numbers of the compounds exemplified refer to those given in the table below, in which the chemical structures and the physical properties of some compounds according to the invention are illustrated.

### EXAMPLE 1

### N-(3-Cyano-5-fluorophenyl)-6-[3-(hydroxymethyl)phenyl]imidazo[1,2-a]pyridine-2-carboxamide

253 μl of a 2M solution of trimethylaluminium in toluene are added dropwise to a solution, cooled to 0° C., of 55 mg of 3-amino-5-fluorobenzonitrile in 1 ml of toluene, followed, after returning to 20° C., by the addition of 100 mg of ethyl 6-[3-(hydroxymethyl)phenyl]imidazo[1,2-a]pyridine-2-carboxylate. The reaction mixture is heated at reflux for 30 hours, then cooled and diluted with 2 ml of water, acidified to pH 3 with N hydrochloric acid and extracted with 5 ml of ethyl acetate and then 5 ml of dichloromethane. The combined organic phases are dried over sodium sulphate, filtered and concentrated under reduced pressure. The residue, combined with the insoluble material isolated at the interface, is washed successively with ethyl ether, ethyl ether/dichloromethane (1/1) and dichloromethane/methanol (99/1) mixtures, and methanol to give 63 mg of N-(3-cyano-5-fluorophenyl)-6-[3-(hydroxymethyl)phenyl]imidazo[1,2-a]pyridine-2-carboxamide in the form of a white solid.

### EXAMPLE 2

### N-(3-Cyanophenyl)-6-(dimethylamino)imidazo[1,2-a]pyridine-2-carboxamide

147 mg of 3-aminobenzonitrile, 474 mg of 1-[bis(dimethylamino)methylene]-1H-1,2,3-triazolo[4,5-b]pyridinium 1-oxide hexafluorophosphate (HATU), 169 mg of 1-hydroxy-7-azabenzotriazole (HOAt) and 424 μl of diisopropylethylamine are added to a solution of 200 mg of 6-dimethylaminoimidazo[1,2-a]pyridine-2-carboxylic acid in 4 ml of N,N dimethylformamide. The reaction mixture is heated at 50° C. for 48 hours, diluted with 3 ml of water and 5 ml of a saturated aqueous sodium hydrogencarbonate solution and stirred for 30 minutes, and then extracted with ethyl acetate. The combined organic phases are dried over sodium sulphate, filtered and concentrated under reduced pressure. The residue is purified by chromatography on silica, the elution being carried out with a gradient of hexane, ethyl acetate and methanol (from 60/37/3 to 0/85/15), to give 61 mg of N-(3-cyanophenyl)-6-(dimethylamino)imidazo[1,2-a]pyridine-2-carboxamide.

### EXAMPLE 3

### Methyl 3-({[6-(dimethylamino)imidazo[1,2-a]pyridin-2-yl]carbonyl}amino)benzoate

265 mg of methyl 3-aminobenzoate are added to a suspension, placed under argon, of 120 mg of 6-dimethylaminoimidazo[1,2-a]pyridine-2-carboxylic acid and 224 mg of 1-(3-dimethylaminopropyl)-3-ethylcarbodiimide hydrochloride in 2 ml of anhydrous pyridine. The reaction mixture is stirred at 50° C. for 48 hours and then concentrated to dryness under reduced pressure. The residue is taken up in 5 ml of chloroform and washed with 2 ml of water. The organic phase is dried over magnesium sulphate and concentrated to dryness under reduced pressure. The residue is purified by chromatography on silica, elution being carried out with a gradient of hexane, ethyl acetate and methanol (from 85/15/0 to 0/85/15), to give 105 mg of methyl 3-({[6-(dimethylamino)imidazo[1,2-a]pyridin-2-yl]carbonyl}amino)benzoate.

The intermediates described below are of use in the preparation of the compounds of the present invention.

### Ethyl 6-dimethylaminoimidazo[1,2-a]pyridine-2-carboxylate

26.2 ml of ethyl bromopyruvate are added to a solution of 19.05 g of 5-dimethylaminopyridine-2-amine (J. Chem. Soc. Perkin 1, 68 (1973)) in 380 ml of dimethoxyethane. The reaction mixture is stirred at 20° C. for 6 hours, then, after addition of 380 ml of ethanol, for 20 hours at reflux and, finally, after cooling, concentrated under reduced pressure. The solid is taken up twice in 350 ml of ethyl ether at reflux and filtered while hot, then twice in 350 ml of ethyl acetate at reflux and filtered while hot, to give 39.66 g of crude ethyl 6-dimethylaminoimidazo[1,2-a]pyridine-2-carboxylate hydrobromide. This salt is taken up in 800 ml of water and treated with solid sodium carbonate, while stirring vigorously, until a pH of 8-9 is reached. The aqueous phase is extracted three times with 500 ml of dichloromethane and the combined organic phases are dried over magnesium sulphate, filtered and concentrated to dryness. The residue is purified by flash chromatography on a silica column, elution being carried out with mixtures of hexane and ethyl acetate (from 5/1 to 1/1), to give 16.7 g of ethyl 6-dimethylaminoimidazo[1,2-a]pyridine-2-carboxylate in the form of a green oil.

¹H NMR spectrum (d₆-DMSO, δ in ppm): 8.35 (s, 1H), 7.81 (d, J=2.2, 1H), 7.45 (d, J=10, 1H), 7.34 (dd, J=2.4, 10, 1H), 4.27 (q, J=7.1, 2H), 2.84 (s, 6H), 1.31 (t, J=7,1, 3H).

### 6-Dimethylaminoimidazo[1,2-a]pyridine-2-carboxylic acid

107 ml of a 2N aqueous lithium hydroxide solution are added at 0° C. to a suspension of 16.7 g of ethyl 6-dimethylaminoimidazo[1,2-a]pyridine-2-carboxylate in a mixture of 220 ml of tetrahydrofuran and 9.5 ml of methanol. The reaction mixture is subsequently reheated to 20° C. and stirred for 4 hours. 2N hydrochloric acid is added dropwise to the reaction mixture, cooled to 0° C., until a pH of 4-5 is reached. The precipitate is filtered off and washed twice with 50 ml of ethyl ether to give 14.8 g of 6-dimethylaminoimidazo[1,2-a]pyridine-2-carboxylic acid in the form of a yellow solid.

¹H NMR spectrum (d₆-DMSO, δ in ppm): 8.67 (s, 1H), 8.18 (d, J=2, 1H), 7.88 (dd, J=2.4, 10, 1H), 7.75 (d, J=10, 1H), 2.96 (s, 6H), (1 acid H not very visible).

### Ethyl 6-[3-(hydroxymethyl)phenyl]imidazo[1,2-a]pyridine-2-carboxylate

475 ml of a mixture of toluene and water (5/1), degassed beforehand, are added, under an argon atmosphere, to a mixture of 25 g of ethyl 6-bromoimidazo[1,2-a]pyridine-2-carboxylate, 13 g of 3-(hydroxymethyl)phenylboronic acid, 5 g of 2-(dicyclohexylphosphino)biphenyl, 1.6 g of palladium acetate and 19 g of potassium phosphate. The reaction mixture is stirred at 80° C. for 16 h and then cooled and diluted with water. After extracting with 2 times 200 ml of dichloromethane, the combined organic phases are dried over sodium sulphate, filtered and concentrated to dryness. The residue is purified by flash chromatography on a silica column, elution being carried out with mixtures of ethyl acetate and methanol (from 100/0 to 96/4), to give 16.1 g of ethyl 6-[3-(hydroxymethyl]phenyl)imidazo[1,2-a]pyridine-2-carboxylate in the form of a light yellow solid.

¹H NMR spectrum (d₆-DMSO, δ in ppm): 8.93 (s, 1H), 8.55 (s, 1H), 7.71-7.66 (m, 3H), 7.57 (d, J=7.7, 1H), 7.48 (t, J=7.6, 1H), 7.39 (d, J=7.5, 1H), 5.29 (t, J=5.7, 1H), 4.61 (d, 5.66, 2H), 4.32 (q, J=7.1, 2H), 1,34 (t, J=7.1, 3H).

### 6-[3-(Hydroxymethyl)phenyl]imidazo[1,2-a]pyridine-2-carboxylic acid

90 ml of a 2N aqueous lithium hydroxide solution are added to a suspension of 17.9 g of ethyl 6-[3-(hydroxymethyl)phenyl]imidazo[1,2-a]pyridine-2-carboxylate in a mixture of 180 ml of tetrahydrofuran and 9 ml of methanol. The reaction mixture is subsequently stirred at 20° C. for 30 minutes. 2N hydrochloric acid is added dropwise to the reaction mixture, cooled to 0° C., until a pH of 4-5 is reached. The precipitate is filtered off and washed twice with 50 ml of ethyl ether to give 15.3 g of 6-[3-(hydroxymethyl]phenyl)imidazo[1,2-a]pyridine-2-carboxylic acid in the form of a white solid.

¹H NMR spectrum (d₆-DMSO, δ in ppm): 8.97 (s, 1H), 8.52 (s, 1H), 7.77-7.67 (m, 3H), 7.57 (d, J=7.7, 1H), 7.48 (t, J=7.6, 1H), 7.39 (d, J=7.5, 1H), 5.7-4.8 (broad s, 1H), 4.60 (s, 2H), (1 acid H not very visible).

The chemical structures (Table 1) and the spectroscopic characteristics (Table 2) of some examples of compounds according to the invention are illustrated in the following tables.

TABLE 1
(I)

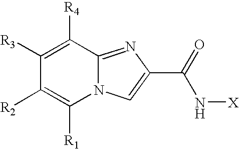

| Ex | R₁ | R₂ | R₃ | R₄ | X |
|---|---|---|---|---|---|
| 01 | H | 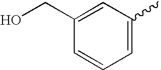 | H | H | 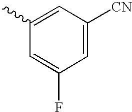 |
| 02 | H | ~NMe₂ | H | H | 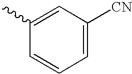 |
| 03 | H | ~NMe₂ | H | H | 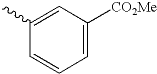 |
| 04 | H | 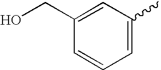 | H | H | 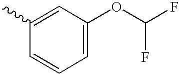 |
| 05 | H | 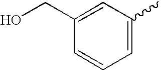 | H | H | 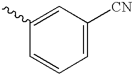 |
| 06 | H | ~NMe₂ | H | H | 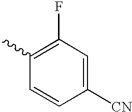 |
| 07 | H | ~NMe₂ | H | H | 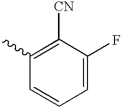 |
| 08 | H | 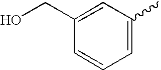 | H | H | 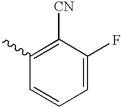 |
| 09 | H | ~NMe₂ | H | H | 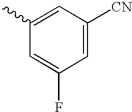 |

TABLE 2

| Ex | Characterizations |
|---|---|
| 01 | ¹H NMR spectrum (d₆-DMSO, δ in ppm): 4.61 (broad d, J = 5.5 Hz, 2H), 5.29 (broad t, J = |
| | 5.5 Hz, 1H), 7.40 (broad d, J = 7.5 Hz, 1H), 7.49 (t, J = 7.5 Hz, 1H), from 7.52 to 7.63 (m, |
| | 2H), 7.69 (broad s, 1H), 7.77 (m, 2H), 8.21 (td, J = 1.5 and 11.5 Hz, 1H), 8.28 (t, J = 1.5 Hz, |
| | 1H), 8.60 (s, 1H), 9.00 (t, J = 1.5 Hz, 1H), 10.95 (s, 1H). |
| | Mass spectrum (LC-MS-DAD-ELSD): m/z 387 [M + H]⁺. |
| 02 | ¹H NMR spectrum (d₆-DMSO, δ in ppm): 2.87 (s, 6H), 7.36 (dd, J = 9.9, 2.5 Hz, 1H), 7.45- |
| | 7.60 (m, 3H), 7.89 (dd, J = 2.5, 1.1 Hz, 1H), 8.21 (dt, J = 7.1 and 2.2 Hz, 1H), 8.33-8.41 (m, |
| | 2H), 10.56 (s, 1H). |
| | Mass spectrum (LC-MS-DAD-ELSD): m/z 304 [M − H]⁻, m/z 306 [M + H]⁺. |
| 03 | ¹H NMR spectrum (d₆-DMSO, δ in ppm): 2.87 (s, 6H), 3.87 (s, 3 H), 7.36 (dd, J = 10.0 and |
| | 2.4 Hz, 1H), 7.47 (t, J = 8.0 Hz, 1H), 7.51 (dt, J = 10.0 and 0.8 Hz, 1H), 7.67 (ddd, J = 8.0, 2.0 |
| | and 1.0 Hz, 1H), 7.90 (dd, J = 2.4 and 0.8 Hz, 1H), 8.06 (ddd, J = 8.0, 2.0 and 1.0 Hz, 1H), |
| | 8.35 (d, J = 0.8 Hz, 1H), 8.66 (t, J = 2.0 Hz, 1H), 10.40 (s, 1H). |
| | Mass spectrum (LC-MS-DAD-ELSD): m/z 339 [M + H]⁺. |
| 04 | ¹H NMR spectrum (d₆-DMSO, δ in ppm): 4.61 (d, J = 5.5 Hz, 2H), 5.29 (t, J = 5.5 Hz, 1H), |
| | 6.90 (dd, J = 2.0 and 7.5 Hz, 1H), 7.21 (t, J = 7.5 Hz, 1H), 7.39 (m, 2H), 7.49 (t, J = 7.5 Hz, |
| | 1H), 7.60 (broad d, J = 7.5 Hz, 1H), 7.69 (broad s, 1H), 7.74 (s, 2H), 7.79 (dd, J = 2.0 and |
| | 7.5 Hz, 1H), 7.90 (t, J = 2.0 Hz, 1H), 8.55 (s, 1H), 8.99 (t, J = 1.5 Hz, 1H), 10.5 (s, 1H). |
| | Mass spectrum (LC-MS-DAD-ELSD): m/z 410 [M + H]⁺. |
| 05 | ¹H NMR spectrum (d₆-DMSO, δ in ppm): 4.60 (d, J = 5.5 Hz, 2H), 5.29 (t, J = 5.5 Hz, 1H), |
| | 7.39 (broad d, J = 7.5 Hz, 1H), 7.49 (t, J = 7.5 Hz, 1H), from 7.52 to 7.64 (m, 3H), 7.69 |
| | (broad s, 1H), 7.76 (m, 2H), 8.24 (m, 1H), 8.40 (m, 1H), 8.59 (s, 1H), 9.00 (t, J = 1.5 Hz, |
| | 1H), 10.75 (s, 1H). |
| | Mass spectrum (LC-MS-DAD-ELSD): m/z 369 [M + H]⁺. |
| 06 | ¹H NMR spectrum (d₆-DMSO, δ in ppm): 2.87 (s, 6H), 7.40 (dd, J = 10.0 and 2.4 Hz, 1H), |
| | 7.57 (dt, J = 10.0 and 0.9 Hz, 1H), 7.74 (ddd, J = 8.4, 2.0 and 1.0 Hz, 1H), 7.87 (dd, J = 2.4 and |
| | 0.9 Hz, 1H), 7.97 (dd, J = 10.9 and 2.0 Hz, 1H), 8.36-8.47 (m, 2H) 9.92 (d, J = 2.8 Hz, 1H). |
| | Mass spectrum (LC-MS-DAD-ELSD): m/z 322 [M − H]⁻, m/z 324 [M + H]⁺. |
| 07 | ¹H NMR spectrum (d₆-DMSO, δ in ppm): 2.88 (s, 6H), 7.30 (m, 1H), 7.39 (dd, J = 10.0 and |
| | 2.4 Hz, 1H), 7.55 (dt, J = 10.0 and 0.9 Hz, 1H), 7.78 (td, J = 8.5 and 6.6 Hz, 1H), 7.86-7.95 |
| | (m, 2H), 8.42 (d, J = 0.9 Hz, 1H), 10.40 (s, 1H). |
| | Mass spectrum (LC-MS-DAD-ELSD): m/z 322 [M − H]⁻, m/z 324 [M + H]⁺. |
| 08 | ¹H NMR spectrum (d₆-DMSO, δ in ppm): 4.60 (d, J = 5.5 Hz, 2H), 5.29 (t, J = 5.5 Hz, 1H), |
| | 7.34 (m, 1H), 7.39 (broad d, J = 7.5 Hz, 1H), 7.49 (t, J = 7.5 Hz, 1H), 7.61 (broad d, J = 7.5 |
| | Hz, 1H), 7.70 (broad s, 1H), from 7.74 to 7.89 (m, 4H), 8.61 (s, 1H), 9.00 (t, J = 1.5 Hz, |
| | 1H), 10.6 (s, 1H). |
| | Mass spectrum (LC-MS-DAD-ELSD): m/z 387 [M + H]⁺. |
| 09 | ¹H NMR spectrum (d₆-DMSO, δ in ppm): 2.87 (s, 6H), 7.37 (dd, J = 10.0 and 2.4 Hz, 1H), |
| | 7.46-7.56 (m, 2H), 7.89 (dd, J = 2.5 and 1.0 Hz, 1H), 8.19 (ddd, J = 11.8, 2.8 and 2.0 Hz, |
| | 1H), 8.24 (t, J = 2.0 Hz, 1H), 8.39 (d, J = 1.0 Hz, 1H), 10.79 (s, 1H). |
| | Mass spectrum (LC-MS-DAD-ELSD): m/z 322 [M − H]⁻, m/z 324 [M + H]⁺. |

The compounds according to the invention have formed the subject of pharmacological trials which make it possible to determine their modulatory effects on NOT.

Evaluation of the In Vitro Activity on N2A Cells

The activity of the compounds according to the invention was evaluated on a cell line (N2A) endogenously expressing the mouse Nurr1 receptor and stably transfected with the NOT binding response element (NBRE) coupled to the luciferase reporter gene. The EC₅₀ values are between 0.01 and 1000 nM. The assays were carried out according to the procedure described below.

The Neuro-2A cell line comes from a standard commercial source (ATCC). The Neuro-2A clone was obtained, from a spontaneous tumour originating from an A albino mouse strain, by R. J Klebe et al. This Neuro-2A line is subsequently stably transfected with 8NBRE-luciferase. The N2A-8NBRE cells are cultured until confluence in 75 cm² culture flasks containing DMEM supplemented with 10% of foetal calf serum, 4.5 g/l of glucose and 0.4 mg/ml of geneticin. After a week of culture, the cells are recovered with 0.25% trypsin for 30 seconds and then resuspended in DMEM without phenol red, containing 4.5 g/l of glucose and 10% of Hyclone delipidized serum, and deposited into transparent-bottom 96-well white plates. The cells are deposited at a rate of 60 000 per well in 75 μl for 24 hours before the addition of the products. The products are applied in 25 μl and incubated for a further 24 hours. On the day of the measurement, an equivalent volume (100 μl) of Steadylite is added to each well and then left for a period of 30 minutes in order to obtain complete cell lysis and maximum signal production. The plates are subsequently measured in a luminescence counter for microplates after having been sealed with an adhesive film. The products are prepared in the form of a stock solution at 10⁻²M and then diluted in 100% of DMSO. Each product concentration is prediluted in culture medium before incubation with the cells, thus containing 0.625% final concentration of DMSO.

For example, compounds Nos. 5 and 8 showed an EC₅₀ value of 27 nM and 0.4 nM respectively.

It is thus apparent that the compounds according to the invention have a modulatory effect on NOT.

The compounds according to the invention can thus be used in the preparation of medicaments for their therapeutic application in the treatment or prevention of diseases involving NOT receptors.

Thus, according to another of its aspects, a subject-matter of the invention is medicaments which comprise a compound of formula (I) or an addition salt of the latter with a pharmaceutically acceptable acid.

According to another of its aspects, a subject-matter of the invention is medicaments which comprise a compound chosen from the compounds of formula (I) as defined above, and also N-(3-chloro-4-cyanophenyl)imidazo[1,2-a]pyridine-2-carboxamide, methyl 3-({[imidazo[1,2-a]pyridin-2-yl]carbonyl}amino)benzoate, methyl 2-({[imidazo[1,2-a]pyridin-2-yl]carbonyl}amino)benzoate, and N-[2-(difluoromethoxy)phenyl]imidazo[1,2-a]pyridine-2-carboxamide, and the addition salts of these compounds with a pharmaceutically acceptable acid.

These medicaments are employed therapeutically, in particular in the treatment and prevention of neurodegenerative diseases, such as, for example, Parkinson's disease, Alzheimer's disease or tauopathies (for example, progressive supranuclear palsy, frontotemporal dementia, corticobasal degeneration or Pick's disease); cerebral traumas, such as ischaemia and cranial traumas and epilepsy; psychiatric diseases, such as schizophrenia, depression, substance dependence or attention deficit hyperactivity disorders; inflammatory diseases of the central nervous system, such as multiple sclerosis, encephalitis, myelitis and encephalomyelitis, and other inflammatory diseases, such as vascular pathologies, atherosclerosis, inflammations of the joints, arthrosis or rheumatoid arthritis; osteoarthritis, Crohn's disease, ulcerative colitis; allergic inflammatory diseases, such as asthma; autoimmune diseases, such as type 1 diabetes, lupus, scleroderma, Guillain-Barré syndrome, Addison's disease and other immune-mediated diseases; osteoporosis; or cancers.

Thus, the present invention is targeted at a compound chosen from a compound of formula (I) as defined above, and also N-(3-chloro-4-cyanophenyl)imidazo[1,2-a]pyridine-2-carboxamide, methyl 3-({[imidazo[1,2-a]pyridin-2-yl]carbonyl}amino)benzoate, methyl 2-({[imidazo[1,2-a]pyridin-2-yl]carbonyl}amino)benzoate, and N-[2-(difluoromethoxy)phenyl]-imidazo[1,2-a]pyridine-2-carboxamide, and the addition salts of these compounds with a pharmaceutically acceptable acid, in the treatment of one of the abovementioned diseases, disorders or conditions.

According to another of its aspects, the present invention relates to the use of a compound chosen from the abovementioned compounds in the preparation of a medicament intended for the treatment and prevention of one of the abovementioned diseases, disorders or conditions.

These compounds might also be used as treatment associated with stem cell transplants and/or grafts.

According to another of its aspects, the present invention relates to pharmaceutical compositions comprising, as active principle, a compound chosen from the group of compounds defined above. These pharmaceutical compositions comprise an effective dose of at least one compound chosen from the group of compounds defined above, and also at least one pharmaceutically acceptable excipient.

The said excipients are chosen, depending on the pharmaceutical form and the method of administration desired, from the usual excipients which are known to a person skilled in the art.

In the pharmaceutical compositions of the present invention for oral, sublingual, subcutaneous, intramuscular, intravenous, topical, local, intratracheal, intranasal, transdermal or rectal administration, the active principle chosen from the abovementioned compounds can be administered in unit administration form, as a mixture with conventional pharmaceutical excipients, to animals and human beings for the prophylaxis or treatment of the above disorders or diseases.

The appropriate unit administration forms comprise oral forms, such as tablets, soft or hard gelatin capsules, powders, granules and oral solutions or suspensions, forms for sublingual, buccal, intratracheal, intraocular or intranasal administration or for administration by inhalation, forms for topical, transdermal, subcutaneous, intramuscular or intravenous administration, forms for rectal administration and implants. For topical application, the compounds according to the invention can be used in creams, gels, ointments or lotions.

By way of example, a unit administration form of a compound according to the invention in the tablet form can comprise the following components:

| | | |
|---|---|---|
| Compound according to the invention | 50.0 | mg |
| Mannitol | 223.75 | mg |
| Croscarmellose sodium | 6.0 | mg |
| Maize starch | 15.0 | mg |
| Hydroxypropylmethylcellulose | 2.25 | mg |
| Magnesium stearate | 3.0 | mg |

There may be specific cases where higher or lower dosages are appropriate; such dosages do not depart from the scope of the invention. According to the usual practice, the dosage appropriate to each patient is determined by the physician according to the method of administration and the weight and the response of the said patient.

The present invention, according to another of its aspects, also relates to a method for the treatment of the pathologies indicated above which comprises the administration, to a patient, of an effective dose of a compound according to the invention or one of its pharmaceutically acceptable salts.

What is claimed is:

1. A compound of formula (I): 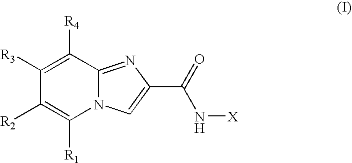
wherein:
X represents a phenyl group substituted by a cyano, a (C₁-C₆)alkoxycarbonyl, a (C₁-C₆)alkoxy substituted by one or more halogens or (C₁-C₆)alkyl substituted by one or more halogens, the phenyl group optionally being substituted a second time by a halogen;
R₁ represents a hydrogen atom, a halogen, a (C₁-C₆)alkoxy group, a (C₁-C₆)alkyl group or an NRaRb group, wherein the alkyl and alkoxy groups may be optionally substituted by one or more halogen, hydroxyl, amino, or (C₁-C₆)alkoxy group;
R₂ represents one of the following groups:
a hydrogen atom,
a (C₁-C₆)alkyl group optionally substituted by one or more groups chosen, independently of one another, from a hydroxyl, a halogen, an amino, an NRaRb group, a (C₁-C₆)alkoxy group and a phenyl group,
a (C₁-C₆)alkoxy group optionally substituted by one or more groups chosen, independently of one another, from hydroxyl, halogen, amino and NRaRb group,
a (C₂-C₆)alkenyl group,
a (C₂-C₆)alkynyl group,
a —CO—R₅ group,
a —CO—NR₆R₇ group,
a —CO—O—R₈ group,
an —NR₉—CO—R₁₀ group,
an —NR₁₁R₁₂ group,
an —N═CH—NRaRb group,
a halogen atom,
a cyano, nitro, hydroxyiminoalkyl or an alkoxyiminoalkyl group,
a (C₁-C₆)alkylthio group,
a (C₁-C₆)alkylsulphinyl group,
a (C₁-C₆)alkylsulphonyl group,
a ((C₁-C₆)alkyl)₃silylethynyl group,
an —SO₂—NR₉R₁₀ group, or
a phenyl group optionally substituted by one or more groups chosen, independently of one another, from the following atoms or groups: halogen, (C₁-C₆)alkoxy, cyano, NRaRb, —CO—R₅, —CO—NR₆R₇, —CO—O—R₈ or (C₁-C₆)alkyl optionally substituted by one or more hydroxyl or NRaRb groups;
R₃ represents a hydrogen atom, a (C₁-C₆)alkyl group, a (C₁-C₆)alkoxy group or a halogen atom;
R₄ represents a hydrogen atom, a (C₁-C₄)alkyl group, a (C₁-C₄)alkoxy group or a fluorine atom;
R₅ represents a hydrogen atom, a phenyl group or a (C₁-C₆)alkyl group;
R₆ and R₇, which are identical or different, represent a hydrogen atom or a (C₁-C₆)alkyl group or form, with the nitrogen atom, a 4- to 7-membered ring optionally including another heteroatom chosen from N, O or S;
R₈ represents a (C₁-C₆)alkyl group;
R₉ and R₁₀, which are identical or different, represent a hydrogen atom or a (C₁-C₆)alkyl group;
R₁₁ and R₁₂, which are identical or different, represent a hydrogen atom or a (C₁-C₆)alkyl group or form, with the nitrogen atom, a 4- to 7-membered ring optionally including another heteroatom chosen from N, O or S;
Ra and Rb represent, independently of one another, a hydrogen atom or a (C₁-C₆)alkyl group or form, with the nitrogen atom, a 4- to 7-membered ring;
with the exception of the compounds where R₁ represents a methyl group or R₂ represents a chlorine atom or R₃ represents a methyl group;
or an acid addition salt thereof;
with the exception of the compounds selected from the group consisting of:
N-(3-chloro-4-cyanophenyl)imidazo[1,2-a]pyridine-2-carboxamide,
methyl 3-({[imidazo[1,2-a]pyridin-2-yl]carbonyl}amino)benzoate,
methyl 2-({[imidazo[1,2-a]pyridin-2-yl]carbonyl}amino)benzoate,
N-[2-(difluoromethoxy)phenyl]imidazo[1,2-a]pyridine-2-carboxamide,
N-[3-(trifluoromethoxy)phenyl]-6-(dimethylamino)imidazo[1,2-a]pyridine-2-carboxamide,
N-[3-(difluoromethoxy)phenyl]-6-(dimethylamino)imidazo[1,2-a]pyridine-2-carboxamide, and
N-[3-(trifluoromethyl)phenyl]-6-(dimethylamino)imidazo[1,2-a]pyridine-2-carboxamide.

2. The compound of formula (I) according to claim 1 wherein X and R₁ to R₄ are as defined in claim 1, wherein at least one of R₁, R₂, R₃ and R₄ is other than a hydrogen atom;
or an acid addition salt thereof;
with the exception of the compounds where R₁ represents a methyl group or R₂ represents a chlorine atom or R₃ represents a methyl group; and
with the exception of the compounds selected from the group consisting of:
N-[3-(trifluoromethoxy)phenyl]-6-(dimethylamino)imidazo[1,2-a]pyridine-2-carboxamide,
N-[3-(difluoromethoxy)phenyl]-6-(dimethylamino)imidazo[1,2-a]pyridine-2-carboxamide, and
N-[3-(trifluoromethyl)phenyl]-6-(dimethylamino)imidazo[1,2-a]pyridine-2-carboxamide.

3. The compound of formula (I) according to claims 1, wherein:
X represents a phenyl group substituted by a cyano, a (C₁-C₆)alkoxycarbonyl or a (C₁-C₆)alkoxy substituted by several halogens, the said phenyl group optionally being substituted a second time by a halogen;
R₂ represents an —NR₁₁R₁₂ group or a phenyl group substituted by a (C₁-C₆)alkyl group itself substituted by a hydroxyl group;
R₁, R₃ and R₄ represent a hydrogen atom;
R₁₁ and R₁₂, which are identical or different, represent a (C₁-C₆)alkyl group;
or an addition salt thereof;
with the exception of the compounds where R₁ represents a methyl group or R₂ represents a chlorine atom or R₃ represents a methyl group; and
with the exception of the compounds selected from the group consisting of:
N-[3-(trifluoromethoxy)phenyl]-6-(dimethylamino)imidazo[1,2-a]pyridine-2-carboxamide and
N-[3-(difluoromethoxy)phenyl]-6-(dimethylamino)imidazo[1,2-a]pyridine-2-carboxamide.

4. The compound of formula (I) according to claim 1, wherein:
X represents a phenyl group substituted by a cyano, CO₂Me or —OCHF₂ and optionally substituted a second time by a fluorine atom;
R₁, R₃ and R₄ represent a hydrogen atom;
R₂ represents an N-dimethyl group or a phenyl group substituted by a hydroxymethyl group;
or an addition salt thereof;
with the exception of N-[3-(difluoromethoxy)phenyl]-6-(dimethylamino)imidazo[1,2-a]pyridine-2-carboxamide.

5. The compound according to claim 1, selected from the group consisting of:
N-(3-cyano-5-fluorophenyl)-6-[3-(hydroxymethyl)phenyl]imidazo[1,2-a]pyridine-2-carboxamide,
N-(3-cyanophenyl)-6-(dimethylamino)imidazo[1,2-a]pyridine-2-carboxamide,
Methyl 3-({[6-(dimethylamino)imidazo[1,2-a]pyridin-2-yl]carbonyl}amino)benzoate,
N-[3-(difluoromethoxy)phenyl]-6-[3-(hydroxymethyl)phenyl]imidazo[1,2-a]pyridine-2-carboxamide,
N-(3-cyanophenyl)-6-[3-(hydroxymethyl)phenyl]imidazo[1,2-a]pyridine-2-carboxamide,
N-(4-cyano-2-fluorophenyl)-6-(dimethylamino)imidazo[1,2-a]pyridine-2-carboxamide,
N-(2-cyano-3-fluorophenyl)-6-(dimethylamino)imidazo[1,2-a]pyridine-2-carboxamide,
N-(2-cyano-3-fluorophenyl)-6-[3-(hydroxymethyl)phenyl]imidazo[1,2-a]pyridine-2-carboxamide, and
N-(3-cyano-5-fluorophenyl)-6-(dimethylamino)imidazo[1,2-a]pyridine-2-carboxamide.

6. A pharmaceutical composition comprising a compound according to claim 1 or a compound selected from N-(3-chloro-4-cyanophenyl)imidazo[1,2-a]pyridine-2-carboxamide, methyl 3-({[imidazo[1,2-a]pyridin-2-yl]carbonyl}amino)benzoate, methyl 2-({[imidazo[1,2-a]pyridin-2-yl]carbonyl}amino)benzoate, and N-[2-(difluoromethoxy)phenyl]imidazo[1,2-a]pyridine-2-carboxamide, or a pharmaceutically acceptable acid addition salt thereof, and also at least one pharmaceutically acceptable excipient.

7. A pharmaceutical composition comprising a compound according to claim 2 or a pharmaceutically acceptable acid addition salt thereof, and also at least one pharmaceutically acceptable excipient.

8. A pharmaceutical composition comprising a compound according to claim 3 or a pharmaceutically acceptable acid addition salt thereof, and also at least one pharmaceutically acceptable excipient.

9. A pharmaceutical composition comprising a compound according to claim 4 or a pharmaceutically acceptable acid addition salt thereof, and also at least one pharmaceutically acceptable excipient.

10. A pharmaceutical composition comprising a compound according to claim 5 or a pharmaceutically acceptable acid addition salt thereof, and also at least one pharmaceutically acceptable excipient.

11. A method for treating or preventing diseases comprising administering to a patient an effective amount of a compound according to claim 1, or a compound selected from N-(3-chloro-4-cyanophenyl)imidazo[1,2-a]pyridine-2-carboxamide, methyl 3-({[imidazo[1,2-a]pyridin-2-yl]carbonyl}amino)benzoate, methyl 2-({[imidazo[1,2-a]pyridin-2-yl]carbonyl}amino)benzoate, and N-[2-(difluoromethoxy)phenyl]imidazo[1,2-a]pyridine-2-carboxamide, or a pharmaceutically acceptable acid addition salt thereof.

12. The method according to claim 11 wherein the disease is a neurodegenerative disease.

13. The method according to claim 11 wherein the disease is cerebral trauma or epilepsy.

14. The method according to claim 11 wherein the disease is a psychiatric disease.

15. The method according to claim 11 wherein the disease is an inflammatory disease.

16. The method according to claim 11 wherein the disease is cancer.

17. The method according to claim 11 wherein the disease is Parkinson's disease, Alzheimer's disease, tauopathies or multiple sclerosis.

18. The method according to claim 11 wherein the disease is schizophrenia, depression, substance dependence or attention deficit hyperactivity disorder.

19. A compound selected from the group consisting of:
Ethyl 6-dimethylaminoimidazo[1,2-a]pyridine-2-carboxylate;
6-dimethylaminoimidazo[1,2-a]pyridine-2-carboxylic acid;
Ethyl 6-[3-(hydroxymethyl)phenyl]imidazo[1,2-a]pyridine-2-carboxylate; and
6-[3-(hydroxymethyl)phenyl]imidazo[1,2-a]pyridine-2-carboxylic acid.
